(19) United States
(12) Patent Application Publication
(10) Pub. No.: US 2003/0175367 A1
(43) Pub. Date: Sep. 18, 2003

(54) Nitrite scavenging formulation to reduce formation of nitrosamines during chewing of food items such as chewing tobacco and betel nuts

(76) Inventor: Hsiang-Kuen Mao, San Jose, CA (US)

Correspondence Address: Hsiang-kuen Mao, 2800 West Pintail Way, Elk Grove, CA, 95758-8201, US

(21) Appl. No.: 10/355,518

(22) Filed: Jan. 31, 2003

(30) Foreign Application Priority Data
Feb. 5, 2002 (TW) 91102034

(51) Int. Cl. A61K 35/78; A61K 38/00; A23L 1/00

(52) U.S. Cl. 424/725; 514/2; 426/531

(57) ABSTRACT

This invention describes a formulation that removes or reduces the formation of nitrosamines when chewing food items such as chewing tobacco, betel nuts, and fruits that contain secondary amines or imines, or other manufactured food items containing sources of amines and imines that could form chemically stable nitrosamines with nitrites in saliva. Nitrosamines are known carcinogens.

### CROSS-REFERENCE TO RELATED APPLICATIONS

Case first filed in Taiwan, Republic of China, Feb. 5, 2002, number 91102034. Also see accompanied case, Table Salt, Table Sugar, And Table Vinegar Formulations with Nitrite Scavengers.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISK APPENDIX.

Not applicable.

### BACKGROUND OF INVENTION

This invention relates generally to a formulation to prevent the formation of stable nitrosamines during chewing of tobaccos, betel nuts and other food items that contain secondary amine derivatives, cyclic imine derivatives or aromatic amine derivatives. Formulations of this invention can be incorporated into chewing tobacco products, chewing nuts such as betel nuts, and other food items that are kept in the mouth for an extended period such as chewing gums, breath mints and hard candies.

Nitrosamines are well known cancer-inducing agents. Nitrosamines are formed by the reaction of an amine, or a cyclic imine, with a nitrosating agent, i.e., a nitrite ion. It is generally believed that the ability of nitrosamines to bind DNA and to alkylate the DNA bases to induce point mutation is responsible in promoting cancer. One such example is the following reaction between guanine, a DNA forming base with N-nitrosodimethyl amine, a nitrosamine:

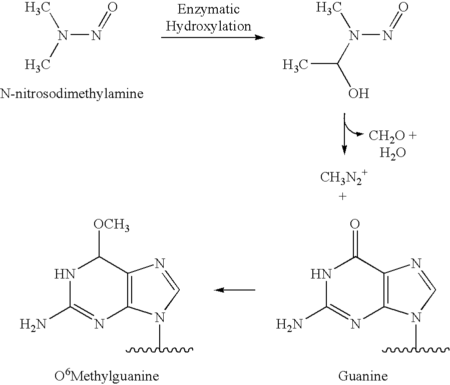

Amine compounds can be classified into primary amines, secondary amines, tertiary amines, aromatic amines and cyclic amines, i.e., imines. Only the secondary amines, imines and aromatic amines react with nitrosating agents to form chemically stable nitrosamines. While the primary amine derived nitrosamines are chemically unstable and decompose quickly. Tertiary amines do not form nitrosamines with nitrosating agents.

The reaction of secondary amines, cyclic amines or aromatic amines with nitrous acid, a nitrosating agent, to give nitrosamines can be represented by the following reaction formula:

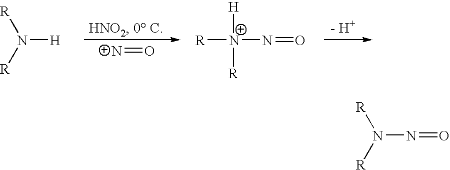

Where Rs can be normal hydrocarbon chains, cyclic chains, aromatic groups, acyclic groups, or the two R groups can be linked in a cyclic structure for an imine.

The reaction between primary amines and a nitrosating agent, nitrous acid in this case, can be given by the following reaction formula:

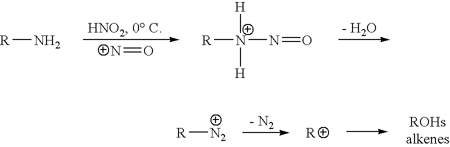

The reaction between tertiary amines and a nitrosating agent give amine salts. These amine salts are not known as cancer inducing agents.

The generally present nitrosating agents are nitrous acid and its salts. These agents are referred to as “nitrites” or “nitrosating agents” in the current writing. The human saliva contains an appreciable amount of nitrites, the nitrosating agent. When in contact with amines, it reacts with these amines to form nitrosamines. With secondary and cyclic amines or aromatic amines, the resulting nitrosamines are chemically stable, and are known carcinogens. With primary amines, the nitrosamines are theorized to take the pathway indicated above to form harmless alcohols. Due to the prolonged resident time in the mouth during chewing, and also due to the abrasion effect of chewing fibrous material that weakens the lining of the oral cavity, it is believed that these chemically stable nitrosamines penetrate into skin and negatively change the DNA of the oral cavity and gum cells to increase the chances of cell mutation and to enhance probability of cancer development.

Nitrate ion is naturally present in drinking water and in food items. It is an essential nutrient to the human body. It has been reported that about 25% of human nitrate intake is excreted by the body into saliva and re-incorporated through gastric juice. Part of this nitrate is reduced to nitrite by bacteria that are present in the mouth. Overall about 5% of the daily nitrate intake is converted into nitrites in saliva. Thus nitrosamine formation in the mouth could take place readily if one eats food items containing secondary or cyclic amines and aromatic amines. For food items that are intended for chewing, i.e. staying in the mouth for a prolonged period of time, the formation of nitrosamine in the mouth tends to be exaggerated, and could account for the increased risk of oral cancer occurrence such as the well published case with chewing tobacco usage, and the prevalent oral cancer associated with the habit of chewing betel nuts in the orient.

It has been reported that antioxidants such as ascorbic acid (vitamin C) can help lower nitrosamine formation in gastric juice thus may help reduce stomach cancer formation. However, it is also known that such antioxidants, when present with nitrate, could lead to enhanced formation of nitrosating agents and the enhanced formation of nitrosamines.

Betel nut is also called Arecae Catechu. The name came from the native language of Malaysia. The meaning of “Catechu” is “a plant juice”. Chewing betel nuts is a popular habit in the areas of Taiwan, Southern China, India, Ceylon, Vietnam, Malaysia, and the Philippines. It is an old habit, believed to have existed over two thousand years. The chewing of betel nuts generates a reaction similar to that of coffee that include increased heartbeat, sweating and an energetic feeling. It has also been reported to be helpful in reducing the symptoms of alcohol intoxication.

Recently the habit of chewing betel nuts has been linked to the development of oral cancer. In the island of Taiwan, about 90% of the patients with oral cancers are regular betel nut chewers. The particular type of oral cancers developed with the chewing of betel nuts carry unmistakable connections, being located mainly between the gum and the oral cavity where one chews the betel nut. Over 50% of all Taiwanese oral cancer patients have these types of cancers. Statistics in the U.S.A. showed that only 14% of all oral cancers are located at the gum and at the surface of the oral cavity.

Detailed studies carried out in Taiwan indicated that several types of chemicals in betel nuts could be responsible for inducing oral cancers: Areca alkaloids such as Arecoline, Arecaidine, Guvacoline, Guvacine, Acolidine; Polyphenolic Compounds,; and Safrole, Eugenol, and Hydroxychavicol etc. The first two classes of compounds are present in the betel nuts. The third class of chemicals is commonly present in the additives. While not all the cancer inducing mechanisms of betel nuts is known, the influence of nitrosamines, derived from the alkaloid amines, are known. (Relevance To Human Cancer Of N-Nitroso Compounds, Tobacco Smoke And Mycotoxins, Edited by I. K. O'NEILL, IARC, Lyon, France, J. CHEN, Chinese Academy of Preventative Medicine, Beijing, People's Republic of China, and H. BARTSCH, IARC, Lyon, France). Another class of known cancer inducing agent is free radicals that are formed due to auto-oxidation. The chewing action that creates abrasion of the gum and the oral cavity lining is also believed to further enhance the penetration of these carcinogens.

The development of oral cancer among chewing tobacco users is well known in the U.S.A. Cancers of the gum and the oral cavity are well known and publicized. While nitrosamine is not the only factor in inducing these oral cancers, it is believed to be an important factor. Within the chewing tobacco there is a abundance of organic amines, i.e., nornicotine, that are ready precursors to the formation of nitrosamines when in contact with nitrosating agents such as the condition existed in saliva during chewing.

Older people with receding gum line in the mouth suffer from teeth sensitivity to eating of hot or cold food and teeth brushing. Special dentifrice products are available to reduce this painful sensitivity. Products such as Sensodyne® toothpaste are one such example. Sodium nitrate or other salts of nitrate is the main active ingredient in these formulations. It is believed that the nitrate ions help numb the teeth by blocking the micro-channels in the teeth. The nitrates are often present at more than 3% in weight of the dentifrice formula. The presence of such a high level of nitrate during teeth brushing, and the natural tendency of nitrate reduction into nitrites in saliva, enhances the concern of introducing nitrites into the digestive system to induce nitrosamines formation and to enhance the possibility of developing cancers of digestive system.

### BRIEF SUMMARY OF THE INVENTION

It is an object of the present invention to provide a formulation for removing or reducing nitrosating agents in saliva, thus help reduce the formation of nitrosamines during chewing food items containing nitrosamines precursors, and, if swallowed, in the gastric digestive system. It is believed that this reduction in nitrosamine formation could help reduce the risk in developing oral cancers and other digestive system cancers.

It is another object of the invention to provide such a method for incorporation of such a nitrite removal formulation into products such as chewing tobaccos, betel nuts, toothpaste containing nitrates, breath mints, hard candies, chewing gums and other food items.

It is also an object of the invention to provide an assured method to reduce human exposure to cancer inducing nitrosamines in the mouth.

### BRIEF DESCRIPTION OF THE SEVERAL VIEWS OF THE DRAWING

Not applicable.

### DETAILED DESCRIPTION OF THE INVENTION

This invention discovered the use of one or more nitrite scavenger additives either alone or in combination with a reducing agent in food products to remove the nitrosating agents in saliva in order to prevent the formation of nitrosamines during chewing.

The preferred scavengers are selected from the class of primary amines and chemicals containing at least one un-substituted amino functional group that could form chemically unstable nitrosamines with nitrosating agents, thus prevent the reaction between these nitrosating agents and the secondary amines and cyclic imines in food items to reduce the formation of cancer-inducing secondary amine and cyclic imine derived nitrosamines. While not intending to be limited by theory, it is believed that the added primary amines competitively react with any nitrosating agents present in saliva to form unstable nitrosamines that are then decomposed to non-carcinogenic chemicals. Thus the addition of these primary amines effectively prevents the formation of carcinogenic nitrosamines that could form from the chemical reaction of secondary amines or cyclic imines with the nitrosating agents in saliva.

The preferred primary amines are those that are acceptable food additives without negative toxicity concerns, as well as being agreeable in taste and smell.

Glucosamine and its salts are the preferred primary amine scavengers of this invention. Glucamines and its salts are also preferred. Examples of the salts include hydrochloride, sulfate, potassium sulfate, sodium sulfate, ascorbate, phosphate, monosodium phosphate, mono-potassium phosphate, di-sodium phosphate, di-potassium phosphate, sodium, potassium phosphate, acetate, lactate, glycolate, citrate, etc.

Glucosamine and its salts are commonly used in health supplement products to reduce joint pains. It is a well-known chemical with a good safety record as a health supplement. It is commonly prepared from seafood shells such as crabs and shrimps. Glucamines and its salts are commonly used as acid-base buffers for intravenous injection medicines, and are commonly prepared by a hydrogenation reaction of ammonia and glucose.

Alternatively, amides of acetic acid, formic acid, propionic acid, isopropionic acid, citric acid, lactic acid, ascorbic acid, glycolic acid, phosphoric acid, gluconic acid etc. of glucosamine or glucamine can also be used. These amides would be more mixable in oily food items, if desired. Saliva action can hydrolyze these amides to give the active primary amines.

Polymeric glucosamines and the acetylated derivative, chitosan and chitin, although somewhat effective, are less preferred due to limited water solubility.

The preferred amino functional group containing chemicals are amino acids. Amino acids of choice are those possessing at least one unsubstituted amino functional group but not containing any sulfur nor sulfide functional groups. Suitable amino acids include glycine, alanine, glutamine, aspartic acid, isoleucine, leucine, lysine, serine, threonine and valine. Salts of these amino acids can also be used if desired.

The nitrosating agents are nitrous acid and its nitrite salts. It has been reported in literature that the human saliva contains from about 6 mg per liter to about 16 mg of nitrites per liter of saliva. During the chewing of chewing tobacco or betel nuts it is estimated that no more than 10 c.c. of saliva is secreted. Thus, in the case of using glucosamine hydrochloride or glucamine hydrochloride as the nitrite scavenger, a level of about 0.00028 gram to about 0.00073 gram of a 100% active glucosamine hydrochloride or glucamine hydrochloride would be required to completely neutralize all nitrites in 10 c.c. of saliva. While not intended to be limited by theory, it is believed that the high water solubility of glucosamine or glucamine enhances its reactivity towards the soluble nitrites, thus is the preferred reactant to react with these nitrites. In practice, an appropriate quantity of the additives should be chewed together with the products containing nitrosamine inducing secondary amines or cyclic imines. In the case of betel nuts, at least 0.00001% by weight of the weight of betel nuts should be added as additive of the primary amines, its salts or its derivatives. Some amount of reducing agent in the forms of either vitamin C or vitamin E should also be incorporated to protect against commonly present free radical reactions. More preferably, in the case of betel nuts, at least 0.001 gram of primary amines, its derivatives, its salts, or a mixture thereof, together with about 0.01 gram of reducing agent in the form of ascorbic acid or vitamin E should be used in each betel nut. To help reduce cost while maintaining effectiveness and good taste, the weight of the additives should be limited to no more than 0.5 gram total weight per betel nut. In the case of chewing tobacco, a solution of glucosamine, glucamine, appropriate amino acids, its salts, its derivatives or a mixture thereof, can be sprinkled

What is claimed is:

1. A food additive nitrite scavenging formula comprising an effective amount of one or more nitrite scavengers.

2. The formulation of claim 1 where the nitrite scavengers are primary amines or compounds with un-substituted amino functional groups.

3. The formulation of claim 1 where the nitrite scavengers further comprise antioxidants.

4. The formulation of claim 2 where the primary amines are selected from the group of glucosamine or glucamine and the corresponding salts.

5. A formulation of claim 2 where the primary amine is in its amide form of acetic acid, formic acid, glycolic acid, citric acid, phosphoric acid, glycolic acid, lactic acid and gluconic acid.

6. A formulation of claim 2 where the compounds with un-substituted amino functional group are amino acids selected from the group of glycine, alanine, glutamine, aspartic acid, isoleucine, leucine, lysine, serine, threonine and valine.

7. A formulation of claim 3 where the antioxidants are Vitamin C, vitamin E, green tea extract, co-enzyme Q-10, garlic extract, lycopene, or other common antioxidants.

8. A chewing tobacco formulation comprising an effective amount of one or more nitrite scavengers.

9. The formulation of claim 8 where the nitrite scavengers are primary amines or compounds with un-substituted amino functional groups.

10. The formulation of claim 8 where the nitrite scavengers further comprise antioxidants.

11. The formulation of claim 9 where the primary amines are selected from the group of glucosamine or glucamine and the corresponding salts.

12. A formulation of claim 9 where the primary amine is in its amide form of acetic acid, formic acid, glycolic acid, citric acid, phosphoric acid, glycolic acid, lactic acid and gluconic acid.

13. A formulation of claim 9 where the compounds with un-substituted amino functional group are amino acids selected from the group of glycine, alanine, glutamine, aspartic acid, isoleucine, leucine, lysine, serine, threonine and valine.

14. A formulation of claim 10 where the antioxidants are Vitamin C, vitamin E, green tea extract, co-enzyme Q-10, garlic extract, lycopene, or other common antioxidants.

15. A betel nut formulation, comprising an effective amount of one or more nitrite scavengers.

16. The formulation of claim 15 where the nitrite scavengers are primary amines or compounds with un-substituted amino functional groups.

17. The formulation of claim 15 where the nitrite scavengers further comprise antioxidants.

18. The formulation of claim 15 where the primary amines are selected from the group of glucosamine or glucamine and the corresponding salts.

19. A formulation of claim 16 where the primary amine is in its amide form of acetic acid, formic acid, glycolic acid, citric acid, phosphoric acid, glycolic acid, lactic acid and gluconic acid.

20. A formulation of claim 16 where the compounds with un-substituted amino functional group are amino acids selected from the group of glycine, alanine, glutamine, aspartic acid, isoleucine, leucine, lysine, serine, threonine and valine.

21. A formulation of claim 17 where the antioxidants are Vitamin C, vitamin E, green tea extract, co-enzyme Q-10, garlic extract, lycopene, or other common antioxidants.

22. A dentifrice formulation for sensitive teeth comprising an effective amount of nitrite scavengers and salts of nitrate.

23. The formulation of claim 22 where the nitrite scavengers are primary amines or compounds with un-substituted amino functional groups.

24. The formulation of claim 22 where the nitrite scavengers further comprise antioxidants.

25. The formulation of claim 22 where the primary amines are selected from the group of glucosamine or glucamine and the corresponding salts.

26. A formulation of claim 22 where the primary amine is in its amide form of acetic acid, formic acid, glycolic acid, citric acid, phosphoric acid, glycolic acid, lactic acid and gluconic acid.

27. A formulation of claim 23 where the compounds with un-substituted amino functional group are amino acids selected from the group of glycine, alanine, glutamine, aspartic acid, isoleucine, leucine, lysine, serine, threonine and valine.

28. A formulation of claim 24 where the antioxidants are Vitamin C, vitamin E, green tea extract, co-enzyme Q-10, garlic extract, lycopene, or other common antioxidants.

29. A hard candy or powdered dip to be used in conjunction with other chewing products like chewing tobacco or betel nuts comprising an effective amount of nitrite scavengers.

30. The formulation of claim 29 where the nitrite scavengers are primary amines or compounds with un-substituted amino functional groups.

31. The formulation of claim 29 where the nitrite scavengers further comprise antioxidants.

32. The formulation of claim 29 where the primary amines are selected from the group of glucosamine or glucamine and the corresponding salts.

33. A formulation of claim 29 where the primary amine is in its amide form of acetic acid, formic acid, glycolic acid, citric acid, phosphoric acid, glycolic acid, lactic acid and gluconic acid.

34. A formulation of claim 29 where the compounds with un-substituted amino functional group are amino acids selected from the group of glycine, alanine, glutamine, aspartic acid, isoleucine, leucine, lysine, serine, threonine and valine.

35. A formulation of claim 31 where the antioxidants are Vitamin C, vitamin E, green tea extract, co-enzyme Q-10, garlic extract, lycopene, or other common antioxidants.
